(19) United States
(12) Patent Application Publication
(10) Pub. No.: US 2023/0202944 A1
(43) Pub. Date: Jun. 29, 2023

(54) Methods for Rapid Formation of Chemicals Including Positron Emission Tomography Biomarkers

(71) Applicant: Fuzionaire, Inc., The Woodlands, TX (US)

(72) Inventors: Anton A. Toutov, Pasadena, CA (US); Sajjad Ghobadi, The Woodlands, TX (US)

(73) Assignee: Fuzionaire, Inc., The Woodlands, TX (US)

(21) Appl. No.: 17/998,785

(22) PCT Filed: Jun. 1, 2021
(86) PCT No.: PCT/US21/35275

§ 371 Date: Nov. 14, 2022

Related U.S. Application Data
(60) Provisional application No. 63/032,372, filed on May 29, 2020.

(51) Int. Cl. C07B 39/00; C07B 59/00

(52) U.S. Cl. CPC C07B 39/00; C07B 59/004; C07F 7/123

(57) ABSTRACT

Methods for rapid, efficient, and safe fluoridation and radiolabeling of established and new biomarkers are described. More specifically, the described herein methods may be used for fluoridation of biomarkers or to facilitate isotopic exchanges, especially ¹⁹F/¹⁸F IEX, for rapid and efficient manufacturing of radiotracers, including radiotracers for positron emission tomography (PET), under clinically relevant conditions.

### CROSS-REFERENCE TO RELATED APPLICATIONS

The present application claims priority under 35 U.S.C. 119(e) to U.S. Provisional Application No. 63/032,372, entitled “Method and Apparatus for the Rapid Formation of Chemicals Including Positron Emission Tomography (PET) Biomarkers” to Toutov, filed May 29, 2020, the disclosure of which is incorporated herein by reference in its entirety.

### FIELD OF THE INVENTION

The invention is generally directed to methods for rapid and efficient synthesis of valuable chemicals, especially of radiotracers for uses in imaging technologies, and especially for rapid and efficient fluoridation and isotope exchange radiofluoridation to produce established and new positron emission tomography (PET) radiotracers.

### BACKGROUND OF THE INVENTION

Biomarkers are biomolecules that indicate (and can be used to trace) biological activity. One example of a biomarker molecule is diacetyl, wherein production and reabsorption of diacetyl by yeast is indicative of a yeast colony's health. More specifically, the health of a yeast colony can be quantitively evaluated by measuring the concentration of diacetyl in media solute samples collected from the colony over time. Furthermore, one particular method of evaluating yeast colonies is to expose them to diacetyl radiolabeled with a radioisotope, and, next, trace the migration of the radioisotope from the radiolabeled diacetyl molecules to the products of yeast metabolism.

Various radiological procedures are used extensively in medicine, including for noninvasive diagnostics. More specifically, various radiation-based medical imaging techniques allow medical professionals to obtain images of internal organs, bones, and tissues of a patient. For example, positron emission tomography (PET) is a noninvasive imaging technique that uses molecules labeled with positron emitting radioisotopes (radiotracers), which, in turn, serve as molecular probes (i.e., biomarkers) for imaging and measuring biochemical processes of mammalian biology in vivo. (M. E. Phelps, Proc. Natl. Acad. Sci. U.S.A. 97, 9226, 2000, the disclosure of which is incorporated herein by reference). PET technique has been proven very useful in the detection of cancer, cardiovascular disease, metabolic disorders, and for imaging of certain structures in the human brain associated with known afflictions, such as Parkinson's and Alzheimer's disease. Although several positron-emitting isotopes, such as ¹⁵O, ¹³N, ¹¹C and ¹⁸F can be used for PET imaging, fluorine-18 (¹⁸F) has proven to be the most clinically relevant radioisotope due to low radiation profile, high spatial resolution, and other factors. For example, the development of radiopharmaceutical [¹⁸F]fluorodeoxyglucose ([¹⁸F]FDG) has profoundly influenced both neuroscience research and clinical diagnosis in oncology.

However, despite favorable properties and proven utility of [¹⁸F]PET, as well as decades of research, methods to efficiently produce functionally and structurally complex [¹⁸F]PET tracers remain scarce. Indeed, although the instrumentation for [¹⁸F]PET is reasonably well established, the method continues to comprise only a small percentage of all clinical imaging activities in healthcare, with [¹⁸F]FDG used in more than 90% of PET procedures. One reason for such deficiency is that ¹⁸F-radiochemistry currently available for ¹⁸F-labeling and production of ¹⁸F-radioisotopes—the tracers with the most favorable nuclear properties—is cumbersome and time consuming. For example, many ¹⁸F-labeling procedures comprise multiple steps and require large precursor quantities and high reaction temperatures, as well as the presence of activating reagents (e.g., strong bases and complexing agents such as expensive cryptands). Consequently, all the additional reaction components lead to unwanted radioactive and chemical side products, which need to be thoroughly separated from the desired, ¹⁸F-labeled tracer prior to administration to a patient. Furthermore, in general, in organic molecules, fluorine atoms are typically attached by a carbon-fluorine bond, yet carbon-fluorine bond formation is challenging, especially in the presence of the variety of functional groups commonly found in structurally complex biologically-relevant molecules, such as, for example, proteins. For PET applications, chemical challenges are exacerbated by the short half-life of ¹⁸F (˜110 minutes), which dictates that carbon-fluorine bond formation occurs at a late stage in the synthesis of the desired radiotracer to avoid unproductive radioactive decay before injection in vivo. Moreover, typically, the source of ¹⁸F for clinical applications is a ¹⁸O (p,n)¹⁸F reaction conducted in a medical cyclotron, which generates aqueous and dilute [¹⁸F]fluorides, and, as such, necessitates water- and high dilution-compatible synthetic methods for generation of the final, desired radiotracer. Therefore, there exist a dire need in the field of PET imaging for effective and inexpensive approaches to facile, clinically viable (e.g., rapid, selective, water-tolerant) radiolabeling with ¹⁸F to produce a variety of desirable radiotracers and radiopharmaceuticals in clinically and or industrially meaningful quantities.

### SUMMARY OF THE INVENTION

Various embodiments are directed to a method for electrospray ionization-assisted fluoridation, including:
providing a target molecule for a fluorine isotope to be installed on and optionally dissolving the target molecule in a first solvent to obtain a target molecule solution;
providing a fluorine anion and dissolving the fluorine anion in a second solvent to obtain a fluoride solution;
mixing the target molecule solution with the fluoride solution to obtain a reaction mixture;
optionally adding one or more reagents for activation of either the fluorine anion, or the target molecule, or both to the reaction mixture;
applying an ionization potential and mode to the reaction mixture, and nebulizing the reaction mixture under the ionization potential and mode to create a plurality of microdroplets comprising the reaction mixture;
allowing the target molecule and the fluorine anion to chemically interact within the plurality of microdroplets to produce a reaction product, wherein the plurality of microdroplets continuously desolvates until collection;
collecting the plurality of microdroplets comprising the reaction product in a collection vessel; to obtain a fluoridation product of the chemical reaction between the target molecule and the fluorine anion in clinical-quality yield and purity.

In various such embodiments, the fluorine anion is selected from the group consisting of: ¹⁹F⁻, ¹⁸F⁻.

In still various such embodiments, the first and second solvent are, independently selected from the solvent list consisting of: water, methanol, ethanol, other polar solvent, other biocompatible solvent known to solubilize biomolecules, dimethylformamide, acetonitrile, another anhydrous solvent, another non-nucleophilic solvent, and any combination thereof.

In yet various such embodiments, the first solvent and the second solvent are the same solvent.

In yet still various such embodiments, the ionization potential is in the range from 2,000 Volts to 7,000 Volts.

In still various such embodiments, the ionization potential is in the range from 3,500 Volts to 7,000 Volts.

In still yet various such embodiments, the ionization mode is selected from the group consisting of: positive, negative.

In yet still various such embodiments, additional means are provided to assist with desolvation of the plurality of microdroplets, and or to guide a flow of the plurality of microdroplets towards the collection vessel, and or to otherwise accelerate the chemical reaction within the plurality of microdroplets.

In yet various such embodiments, the additional means comprise one or more means selected from the list consisting of: utilizing a nebulizing gas heated to 25-300° C.; heating or cooling the reaction mixture at any point of the reaction; condensing, quenching, and or humidifying the plurality of microdroplets at collection; and any combination thereof.

In yet still various such embodiments, the collected plurality of microdroplets are purified to isolate the reaction product prior to use.

In still various such embodiments, the target molecule is a Fluoride Acceptor comprising a linker moiety for conjugation with a final target.

In still yet various such embodiments, the Fluoride Acceptor is a prosthetic molecule selected from the group consisting of: a SiFA, a HetSiFA.

In yet various such embodiments, the target molecule comprises a Fluoride Acceptor functionality.

In still yet various such embodiments, the Fluoride Acceptor functionality is a prosthetic functionality selected from the group consisting of: a SiFA functionality, a HetSiFA functionality.

Additional embodiments and features are set forth in part in the description that follows, and in part will become apparent to those skilled in the art upon examination of the specification or may be learned by the practice of the disclosed subject matter. A further understanding of the nature and advantages of the present disclosure may be realized by reference to the remaining portions of the specification and the drawings, which form a part of this disclosure.

### BRIEF DESCRIPTION OF THE DRAWINGS

These and other features and advantages of the present invention will be better understood by reference to the following detailed description when considered in conjunction with the accompanying data and figures, wherein:

FIG. 1 provides a schematic depiction of clinical PET imaging with a radiotracer according to prior art.

FIG. 2 provides examples of ¹⁸F-labeled prosthetic molecules featuring various probe linkers for facile installation onto radioprobes of interest, according to prior art.

FIG. 3 illustrates radiolabeling of biotin via B- and Si-chemistries of the corresponding pre-installed prosthetic groups, according to prior art.

FIG. 4 provides examples of silicon fluoride acceptor (SiFA) prosthetic molecules with various probe linkers, and illustrates radiolabeling thereof via isotopic exchange (IEX) according to prior art.

FIG. 5 provides examples of heterocyclic SiFA (HetSiFA) moieties, and illustrates synthesis and IEX radiolabeling of HetSiFA prosthetics, according to prior art.

FIG. 6 provides a schematic illustrating an apparatus and process for conducting an electrospray ionization (ESI)-assisted chemical reaction, according to prior art.

FIG. 7 schematically illustrates the process for ESI- and HetSiFA-assisted radiofluoridation, wherein R represents either a linker functionality for post-fluoridation linking to the probe/biomarker of interest, or the probe/biomarker itself, in accordance with embodiments of the application.

FIG. 8 schematically illustrates the process for ESI- and HetSiFA-assisted, late stage radiofluoridation of a biomarker for PET imaging, in accordance with embodiments of the application.

FIG. 9 illustrates ESI-assisted, direct radiofluoridation of fluorodeoxyglucose, in accordance with embodiments of the application.

FIGS. 10A through 10D schematically illustrate the process for ESI-assisted direct fluoridation or radiofluoridation of various classes or organic functionalities, wherein FIG. 10A illustrates fluoridation or radiofluoridation of an aliphatic functionality; FIG. 10B illustrates fluoridation or radiofluoridation of an aromatic or heteroaromatic functionality; FIG. 10C more specifically illustrates fluoridation or radiofluoridation of a pyridine; and FIG. 10D more specifically illustrates fluoridation or radiofluoridation of an indole; wherein R represents either a linker functionality for post-fluoridation linking to the probe/biomarker of interest, or the probe/biomarker itself; X represents a labile leaving group displaceable by a F⁻ anion; and ⁹¹F⁻/¹⁸F⁻ are used interchangeably, in accordance with embodiments of the application.

FIG. 11 provides examples illustrating the ESI-assisted fluoridation in accordance with embodiments of the application.

### DETAILED DISCLOSURE

The embodiments of the invention described herein are not intended to be exhaustive or to limit the invention to precise forms disclosed. Rather, the embodiments selected for description have been chosen to enable one skilled in the art to practice the invention.

As used herein, the terms “imaging agent,” “imaging probe,” or “imaging compound,” means, unless otherwise stated, a molecule which can be detected by its emitted signal, such as positron emission, autofluorescence emission, or optical properties. The method of detection of the compounds may include, but are not necessarily limited to, nuclear scintigraphy, positron emission tomography (PET), single photon emission computed tomography (SPECT), magnetic resonance imaging, magnetic resonance spectroscopy, computed tomography, or any combination thereof, depending on the intended use and the imaging methodology available to the medical or research personnel.

As used herein, the terms “biomolecule” and “biomarker” refer to any molecule produced or used by a living organism and may be selected from the group consisting of, but not limited to: a ligand, a disease targeting molecule, a peptide, a protein, an enzyme, an antibody, a small molecule, a polymer, a polysaccharide, a carbohydrate, a lipid, as well as any analog, including synthetic analogs, or fragments thereof.

As used herein, “radioisotope” is any atomic isotope or radioactive nuclide/radionuclide that may be used for radiolabeling. The invention disclosed herein may be practiced with various radioisotopes to produce biomarkers and other chemicals useful in various medicinal and non-medicinal fields. In some embodiments, the radioisotope of interest is ¹⁸F, especially as useful for PET applications. However, in other embodiments, ²¹²Bi, ¹³¹I, ²⁴Na, or another radioisotope may be utilized to produce, according to the instant methods, valuable chemicals for medical, industrial, and or research applications.

As used herein, “fluorination” encompasses incorporating fluorine by any mechanism, including, but not limited to, electrophilic, nucleophilic, and radical fluorinations. In turn, “fluoridation” refers to the use of fluoride (i.e., fluorine anion) for incorporation of fluorine atom into a molecule. This means that fluorination includes fluoridation—i.e., fluoridation chemical methods are a subset of fluorination chemical methods.

Turning now to the schemes, images, and data, embodiments of methods for efficient, rapid, and safe fluoridation and radiofluoridation of high value biomarkers, or other organic- or bio-molecules, are described. However, in some embodiments, the methods are used to install another halogen, that is not fluorine, or another functional group. In some embodiments, the high value biomarkers are established radiotracers for PET or other imaging applications, while in many other embodiments, the methods of the instant disclosure enable design and manufacture of novel radiotracers. In many embodiments, the methods afford reaction conditions that are relevant to both research and clinical applications, such as, for example, biologically-relevant temperatures and aqueous reaction media, and afford the desired products in clinically-significant yields and amounts. More specifically, in many embodiments, the methods provide unique reaction conditions, which induce acceleration of the desired reaction rates, as compared to the rates of the same reactions run in bulk solution. In some embodiments, a biomarker of choice is outfitted with a fluoridation prosthetic moiety prior to application of one of the instant methods, wherein such method targets the pre-installed fluoridation prosthetic for fluoridation or radiofluoridation. In some embodiments, the fluoridation prosthetic moiety is first fluoridated or radiofluoridated via one of the instant methods and only then installed onto the biomarker to complete the biomarker's labeling with one of fluoride's isotopes. In many such embodiments utilizing prosthetic moieties, the instant methods afford ultrafast fluoridation kinetics. In some embodiments, prosthetic moieties for addition of other isotopes or functional groups are used in either of the ways disclosed herein. In some embodiments, radiofluoridation or radiolabeling of the instant methods is an isotopic exchange (IEX) accelerated by the instant methods, as compared to the same IEX conducted in a bulk solution.

PET using fluorine-18 radioisotope promises to become the foremost nuclear imaging methodology for clinical diagnostic medicine, as well as research, due to its many favorable properties. In particular, [¹⁸F]PET radiopharmaceuticals exhibit favorable radiation profile and high imaging sensitivity. In addition, PET radiotracers can be designed to have extremely high selectivity for targeting specific diseases and in vivo processes. However, despite many advantages of [¹⁸F]PET application to either a whole body imaging or imaging of specific target structures, this technique also possesses important limitations. Indeed, although the instrumentation for [¹⁸F]PET is reasonably well established, the method continues to comprise only a small percentage of all clinical imaging activities in healthcare. As such, it appears that one of the primary impedances to the widespread use of [¹⁸F]PET as imaging modality is the limitations of fundamental chemical synthesis in the production of new suitable ¹⁸F-radiopharmaceuticals.

More specifically, the main synthetic challenge in the development of new [¹⁸F]PET agents, is the installation of ¹⁸F atom onto a biomolecule to efficiently and cleanly generate the desired radiochemical probe. This may, at first, appear somewhat surprising given the number of efficient and selective fluorination methods available to the scientific community, especially those developed and reported in recent years. (See, for example: Liang, T; Neumann, C. N.; Ritter, T. Angew. Chem. Int. Ed. 2013, 52, 8214; Champagne, P. A.; Desroches, J.; Hamel, J.-D.; Vandamme, M.; Paquin, J.-F. Chem Rev. 2015, 115, 9073; and Hollingworth, C.; Gouverneur, V. Chem. Commun. 2012, 48, 2926; the disclosure of all of which is incorporated herein by reference.) However, it is well appreciated in the field of [¹⁸F]PET that the fluorination methodologies developed for the installation of abundant “cold” fluorine ¹⁹F onto organic molecules are rarely amenable to efficient installation of radioactive fluorine ¹⁸F onto biologically relevant targets. Indeed, typical fluorination strategies employ electrophilic fluorine sources, heating, non-aqueous reaction conditions, and lengthy reaction times (often followed by time-consuming chromatographic purification) incompatible with the 109.8-minute half-life of ¹⁸F radioisotope. Therefore, it is crucial for the clinical relevance of any new PET ¹⁸F-radiotracer that the installation of the ¹⁸F radioisotope occurs by a fast, convenient (e.g., aqueous), and highly efficient (including with minimal, if any, side products) fluorination methodology, preferably immediately prior to PET agent administration (i.e., at the final stages of the synthesis). Unfortunately, such direct fluorination methods remain largely elusive.

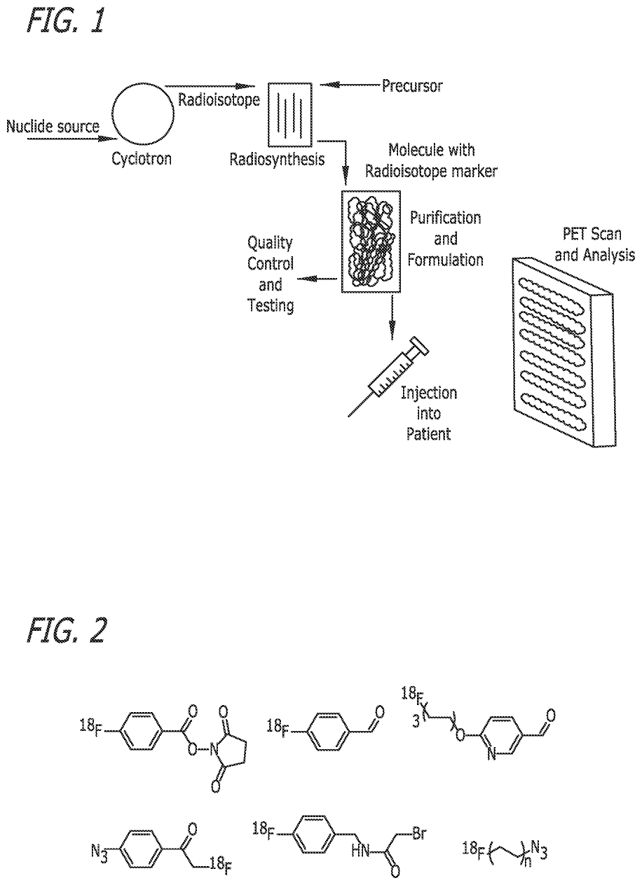

Typically, medical ¹⁸F-radioisotope is separately produced in a cyclotron and then reacted with another molecule to obtain the desired radiopharmaceutical (FIG. 1). It is often impossible to directly combine these steps because most bio-relevant molecules (i.e., molecules with radiopharmaceutical potential) would be destroyed by the cyclotron process. For example, many desirable radiopharmaceuticals, such as, for example, many proteins and antibodies, are heat sensitive, and, therefore, cyclotron reaction temperatures may irrecoverably change their molecular characteristics, and or result in very poor decay-corrected radiochemical yields.

However, a number of indirect labeling methods, such as methods based on the concept of a prosthetic group, have been developed in order to circumvent synthetic and handling challenges associated with direct installation of ¹⁸F-radioisotope onto molecular probes of interest. The prosthetics approach is especially advantageous in the installation of a radioisotope onto complex bioactive molecules, such as peptides or proteins, which comprise many sensitive groups and functionalities that are potentially incompatible with fluorination chemistries. According to such approach, a simpler and/or smaller chemical entity is first manipulated to either directly display ¹⁸F or to be pre-activated for facile radiolabeling at a later time, and then straightforwardly incorporated into the probe of interest via a suitable pre-installed linker.

Some examples of radiolabeling prosthetics featuring a pre-installed ¹⁸F and a probe conjugation-enabling linker are shown in FIG. 2. The suitable linkers may, for example, include linkers featuring N-succinimidyl functionality for conjugation to probe's primary amines, or azide functionality for cycloaddition conjugation to probe's alkyne functionality, or other linkers. Some such probes and methods are described by Liu et al., 2011, Mol. Imaging 10:168; Cai et al., 2007, J. Nucl. Med. 48:304; or Olafsen et al., 2012, Tumor Biol. 33:669; the disclosures of which are incorporated herein by reference. As a more specific example, [18F]N-succinimidyl fluorobenzoate (¹⁸F-SFB) in FIG. 2 is a radiolabeled prosthetic group which readily reacts with ε-amino groups of protein surface-exposed lysine residues, and, therefore, can be used to radiolabel such proteins or peptides with ¹⁸F. However, radiolabeling with ¹⁸F-SFB suffers from poor selectivity and poor overall decay-corrected radiochemical yield (1.4-2.5%, after 3-step synthesis of ¹⁸F-SFB itself, followed by protein conjugation). As another example, site-specific radiolabeling of proteins using 4-¹⁸F-fluorobenzaldehyde (¹⁸F-FBA, also depicted in FIG. 2) has also been demonstrated.

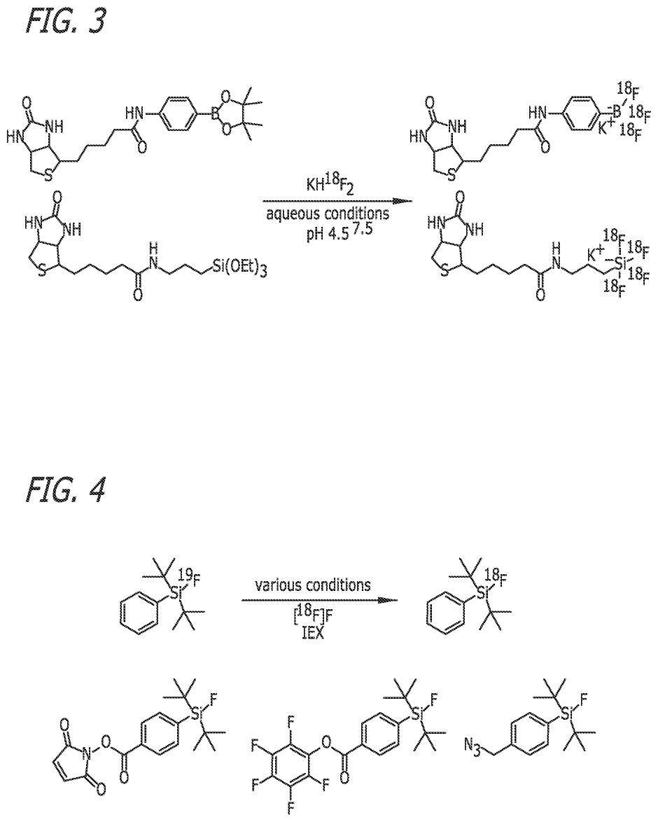

Alternatively, some ¹⁸F-radiolabeling strategies rely on prosthetic molecules called fluoride acceptors (FAs), which comprise a molecular site with less demanding bond forming chemistry between F atom and a non-carbon atom (e.g., boron or silicon—FIG. 3). For example, FIG. 4 provides examples to illustrate the concept of organosilicon-based prosthetics for introduction of radiofluoride by nucleophilic displacement at a silicon atom, also referred to as silicon fluoride acceptors, or SiFAs. This approach has been gaining significant interest in the area of ¹⁸F-labeling (see, for example: Wangler, C.; Kostikov, A; Zhu, J.; Chin, J.; Wangler, B; Schirrmacher, R. Appl. Sci. 2012, 2:277-302; Bernard-Gauthier, V.; et al. BioMed Research Int., 2014, 20; the disclosures of which are incorporated herein by reference). Many appeals of SiFA labeling strategies include fast, yet mild, aqueous reaction conditions, which solve a variety of limitations inherent to most C-F radiofluorination methods, including many C-F prosthetic group methods. However, the most attractive aspect of SiFA prosthetics is that they allow for a scenario illustrated in FIG. 4, wherein an organic molecule already containing a Si-¹⁹F bond can be radiolabeled via [¹⁹F]/[¹⁸F] isotopic exchange (IEX). Therefore, SiFA strategies allow for late stage radiofluorination, wherein the desired ¹⁸F-containing molecular probe can be produced in a single step without any of the tedious subsequent chromatographic purification steps associated with other radiolabeling methods.

Unfortunately, despite many advantages of SiFA labeling methods, they also suffer from a number of drawbacks. Most importantly, the Si—F bond, although thermodynamically stable, is kinetically unstable under physiological conditions, where it is susceptible to destructive nucleophilic attacks by water molecules. Accordingly, very bulky hydrophobic tert-butyl (tBu) groups are commonly used to shield and protect the Si—F bond of SiFAs from water. However, these hydrophobic groups also impart extreme lipophilicity to SiFAs, which is a problem for diagnostic/PET molecular probes because it often triggers the hepatic first pass effect. As such, probes featuring lipophilic SiFAs are very susceptible to metabolism and destruction prior to reaching their intended targets, which, in turn, severely lowers their bioavailability and tremendously minimizes their diagnostic value. These lipophilicity setbacks are especially difficult to overcome for the simple aromatic phenyl-type SiFAs, which are standard aromatic SiFA fragments in silicon radiochemistry. Consequently, the challenges associated with the currently available SiFA-based radiotracers include poor in vivo stability and unfavorable pharmacokinetic behavior. Notably, similar problems plague attempts to exploit B—F, S—F, Al—F, and P—F bonds (instead of C—F and Si—F bonds) to create novel FAs. Organofluorines, such as organotrifluoroborates have been also investigated as potential approaches to ¹⁸F-radiolabeling, however, the production of these desirable molecules has been slow and inefficient.

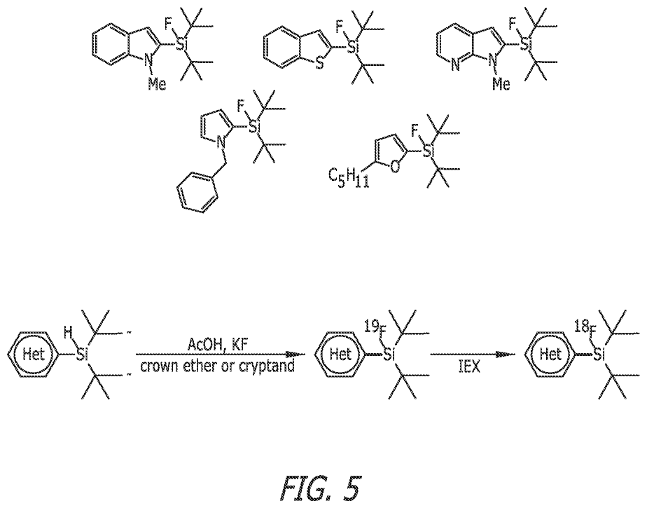

Nevertheless, a recently reported class of SiFAs—heterocyclic SiFAs (HetSiFAs), which feature a heterocyclic core (i.e., a monocyclic or bicyclic heteroaryl ring), promises to alleviate many of the challenges associated with other SiFAs, especially the phenyl SiFAs, including a more straightforward preparation and improved pharmacokinetic properties. More specifically, Waldmann et. al. have recently reported HetSiFA prosthetics (FIG. 5), wherein a silicon fluoride acceptor functionality Si(tBu)₂F is directly attached to an aromatic heterocycle via a carbon-silicon bond (WO 2016/191424). As such, HetSiFAs promise to overcome some of the challenges associated with production and radiolabeling of ¹⁸F-radiopharmaceuticals, especially the challenges associated with difficult and variability-limiting syntheses, radiolabeling efficiency, probe stability, and lipophilicity. In particular, with respect to lipophilicity, HetSiFAs offer increased molecular polarity, as compared to traditional phenyl-based SiFAs, due to the heterocycle's heteroatom. Increased polarity, in turn, decreases the tendency of small probes to be subjected to the hepatic first pass effect and untimely metabolic loss in vivo. Furthermore, the presence of certain heteroatoms, such as nitrogen, allows for additional water solubilizing tactics, such as quaternarization of a nitrogen, or easy functionalization with various hydrophilic functionalities. In addition, the HetSiFA's heteroatom or heteroatoms can also be used for facile diversification of molecular PET tracers, including via bioconjugation to a peptide or antibody of interest. Moreover, some HetSiFAs offer various advantages over their simpler phenyl SiFA analogs due to unique steric and electronic features. For example, HetSiFAs have demonstrated unprecedented radiolabeling speed and efficiency in IEX ¹⁸F radiolabeling studies.

In addition, advantages of using HetSiFA prosthetics for ¹⁸F-radiolableing include straightforward synthesis of HetSiFA moieties, which often allow to avoid the use of highly pyrophoric lithium or magnesium reagents (often required for installation of F atom by other synthetic methods), or pre-functionalization of the aryl. Accordingly, radiolabeling with HetSiFAs has great potential for scalability, including to industrially-meaningful amounts, sustainability, and “green” efficiency (resulting from reagents economy). Moreover, the great variety of available heteroaromatic compounds that can serve as a HetSiFA core allow for virtually unlimited diversification of radiofluoridation prosthetics and, as a result, of radiolabeled biomarkers, differing in chemical structure, polarities, and types of derivatization.

Electrospray ionization (ESI) is a technique in which high voltage is applied to a liquid to produce aerosol ions. ESI is commonly used in mass spectrometry (MS), colloid thrusters, polymer coating, nanospray direct writing, and the paint industry. However, ESI is a particularly useful technique for activating molecules for analysis by MS systems, because it overcomes the propensity of these molecules, especially macromolecules, to fragment when ionized. In fact, ESI is called ‘soft ionization’ technique, because it causes very little molecular fragmentation. Typically, during an ESI process, a solution comprising molecules of interest is ionized by application of high electrical current and vaporized to produce ES droplets, wherein the resulting charged droplets may be next forwarded to a MS system for mass spectroscopy, or otherwise collected and analyzed.

Since early 2000s, ESI methods have been used as a sample preparation technique, mostly for MS, which can produce micro and sub-micrometer scale droplets with charged surfaces. Moreover, it has been shown, that reacting chemical reagents via such droplets has a potential to accelerate various chemical reactions by multiple orders of magnitude, especially as compared to the same reactions conducted in bulk solution (see, for example: Yan, X., et al., “Organic Reactions in Microdroplets: Reaction. Acceleration Revealed by Mass Spectrometry,” Angewandte Chemie International Edition, 2016, 55, 12960-12972, the disclosure of which is incorporated herein by reference). Not to be bound by theory, the demonstrated dramatic rate accelerations are believed to be the consequence of the high surface area localizations afforded by the droplets for chemical reactions to occur on. Furthermore, other potential benefits of applying ESI techniques to chemical synthesis include: advantages of miniaturization (wherein today's microfluidic systems effectively enable the compartmentalization of reactions to as low as nanoliter volumes), lesser reagent consumption, high throughput device fabrication, rapid mixing of reagents, careful real-time control and monitoring of reaction conditions, opportunities for automation and programmable control, such as that observed in flow chemistry devices.

As such, the capability of ESI technique to promote and or accelerate various chemical reaction is being extensively investigated. For example, the following organic chemistries have been reported to benefit from application of ESI: Michael addition (Girod, M. et al., Accelerated bimolecular reactions in microdroplets studied by desorption electrospray ionization mass spectrometry. Chemical Science 2011, 2 (3), 501-510, the disclosure of which is incorporated herein by reference), Schiff base formation (Bain, R. M., et al., Accelerated hydrazone formation in charged microdroplets. Rapid Communications in Mass Spectrometry 2016, 30 (16), 1875-1878, the disclosure of which is incorporated herein by reference), Claisen-Schmidt condensation (Müller, T., et al., Accelerated Carbon-Carbon Bond-Forming Reactions in Preparative Electrospray. Angewandte Chemie International Edition 2012, 51 (47), 11832-11835, the disclosure of which is incorporated herein by reference), amide formation (Gibard, C., et al., Phosphorylation, oligomerization and self-assembly in water under potential prebiotic conditions. Nature Chemistry 2018, 10 (2), 212-217; and Wleklinski, M., et al., Can Accelerated Reactions in Droplets Guide Chemistry at Scale? European Journal of Organic Chemistry 2016, 2016 (33), 5480-5484, the disclosures of which is incorporated herein by reference), ester hydrolysis (Banerjee, S., et al., Can all bulk-phase reactions be accelerated in microdroplets? Analyst 2017, 142 (9), 1399-1402, the disclosure of which is incorporated herein by reference), synthesis of nanoparticles (Li, A., et al., Synthesis and Catalytic Reactions of Nanoparticles formed by Electrospray Ionization of Coinage Metals. Angewandte Chemie International Edition 2014, 53 (12), 3147-3150, the disclosure of which is incorporated herein by reference), Hantzsch reaction (Bain, R. M., et al., Accelerated Hantzsch electrospray synthesis with temporal control of reaction intermediates. Chemical Science 2015, 6 (1), 397-401, the disclosure of which is incorporated herein by reference), Biginelli reaction (Sahota, N., et al., A microdroplet-accelerated Biginelli reaction: mechanisms and separation of isomers using IMS-MS. Chemical Science 2019, 10 (18), 4822-4827, the disclosure of which is incorporated herein by reference), hydrogen-deuterium exchange (Jansson, E. T., et al., Rapid Hydrogen-Deuterium Exchange in Liquid Droplets. Journal of the American Chemical Society 2017, 139 (20), 6851-6854, the disclosure of which is incorporated herein by reference), pinacol rearrangement (Chen, H., et al., Organic Reactions of Ionic Intermediates Promoted by Atmospheric-Pressure Thermal Activation. Angewandte Chemie International Edition 2008, 47 (18), 3422-3425, the disclosure of which is incorporated herein by reference), organometallic reactions (Iyer, K., et al., Accelerated multi-reagent copper catalyzed coupling reactions in micro droplets and thin films. Reaction Chemistry & Engineering 2018, 3 (2), 206-2092, the disclosure of which is incorporated herein by reference). However, although there exist multiple reports investigating ESI effect on accelerating and or promoting various chemical reactions, such reports, typically, only track and analyze the reaction progression/kinetics via, for example, MS, and none suggests using ESI to deliberately drive a given reaction towards a specific desired product, or further adjusting conditions of such ESI-assisted reactions in order to maximize the yield of the desired product, and, most importantly, none reports collecting such product in meaningful amounts for further applications. In other words, no report to date has suggested using an ESI setup as a purely synthetic/standalone module.

This application is directed to embodiments of methods for rapid, efficient, and safe, electrospray-assisted fluoridation and or radiofluoridation of established and new biomarkers for various applications, including for diagnostic imaging. However, in some embodiments, the instant methods are used to install a halogen isotope other than ¹⁸F/¹⁹F, or another functionality. In many embodiments, the methods rely on electrospray ionization (ESI). In many such embodiments, the methods provide mild reaction conditions compatible with clinical applications, including mild reaction temperatures and aqueous reaction media. In many embodiments, the methods afford [⁹F]- or [¹⁸F]-labeled biomarkers or otherwise radiolabeled radiotracers for applications in, for example, PET imaging. In some embodiments, the methods comprise outfitting a biomarker of choice with a prosthetic moiety amenable to facile and targeted installation of a fluorine atom or a radioisotope of choice, prior to subjecting the biomarker to the ESI-assisted fluoridation or radiolabeling. In other embodiments, such prosthetic moiety is first fluoridated or radiolabeled according to the methods of the instant disclosure and only then attached to the biomarker of choice. In many embodiments the prosthetic moiety comprises one of the functionalities selected from the list consisting of: a SiFA functionality, a HetSiFA functionality. In many embodiments, the methods of the instant disclosure offer substantial reaction rate acceleration, as compared to the currently available fluoridation and radiolabeling methods.

Although the instantly disclosed methods are particularly useful for fluoridation and radiofluoridation of organic molecules, and especially for IEX-type radiofluoridation, in some embodiments, the instant methods are used to install stable (non-radioactive) and radioactive isotopes of elements other than fluorine onto the biomarkers (or other chemicals) of choice, still taking advantage of the methods' excellent reaction rates and safety. For example, in some embodiments, the instant methods are used for non-fluorine halogenation of molecules, for example for installation of an iodine isotope. In many embodiments, the methods are used to rapidly and efficiently form bonds between a fluorine atom, or another halogen, including any isotope thereof, and an atom selected from the list (but not limited to): carbon, silicon, boron, sulfur, aluminum, and phosphorus.

In many embodiments, the instant methods enable and or accelerate chemical reactions by turning the solution reaction media into accelerated, desolvated microdroplets. In many such embodiments, the microdroplets are generated via application of electrospray ionization (ESI). In many embodiments, activating and or accelerating the reaction components via electrospray microdroplets allows to avoid additional heating for reagent activation, which is in contrast to most currently available batch processes. Accordingly, in many embodiments, the reaction conditions afforded by the instant methods are very mild and compatible with thermally-sensitive biomolecules, such as many antibodies and proteins. Furthermore, in many embodiments, microdroplets of embodiments formed by the ESI process undergo continuous and rapid solvent evaporation (desolvation) as they move towards collection, which, in turn, increases reagents' concentrations and, thus, greatly enhances reactions' efficiencies, especially as compared to, for example, ¹⁸F-labeling batch reaction processes typically conducted in very dilute solutions, rendering final stage radiofluoridation under standard batch or microfluidic conditions too slow to be effective.

In many embodiments, the methods afford preparative scale syntheses. In many embodiments the methods produce up to milligram quantities of desired products. In many embodiments, the methods produce clinically sufficient amounts of desired products, sufficient, for example, for relevant clinical imaging applications, such as, for example, PET imaging.

In many embodiments, the synthetic procedures enabled and or accelerated by the methods of the instant application install an atom or another chemical functionality onto a biomarker. In many embodiments, the synthetic procedures are radiolabeling, and the atom or another chemical functionality is a radioisotope to turn the biomarker of choice into a radiotracer for imaging applications. In many embodiments the radiolabeling is, more specifically, a radiofluoridation, and the radioisotope is ¹⁸F.

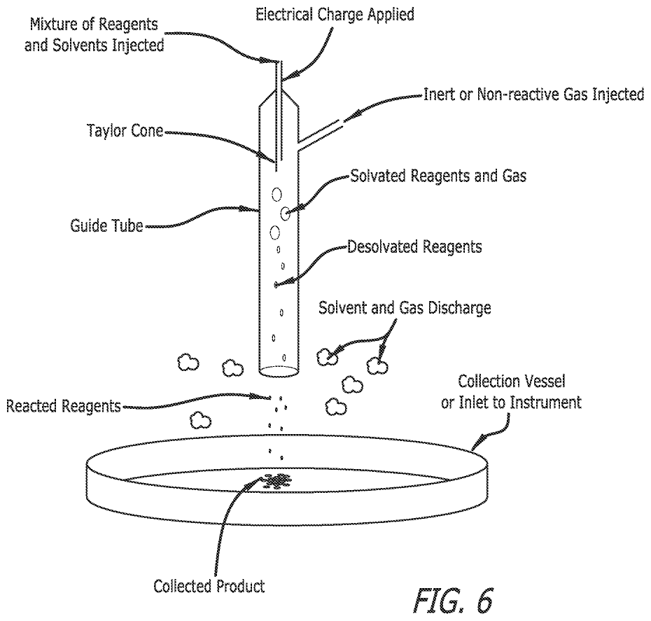

FIG. 6 schematically illustrates a typical ESI apparatus for use with the ESI-assisted synthetic methods of many embodiments. It should be noted, that the elements and features of the apparatus in FIG. 6 are not, necessarily, drawn to scale, including relative to each other, nor are all of the elements and features of the apparatus are required for accomplishing the instant methods, but FIG. 6 is only meant to show the relative processes. In many embodiments, the apparatus comprises: a capillary tube with an inlet/injection site for the reagents to be reacted, a high voltage source for applying high voltage to the reaction solution in the capillary tube, a guide tube enclosing the capillary tube, wherein the guide tube may have additional inlets as needed, and a receptacle/collection vessel for collecting the products of the reaction. Accordingly, in many embodiments of the methods of the instant application, the solution of the reagents to be reacted in a desired way is injected into and passed through the capillary tube, where it is charged/ionized, and, next, ejected through the opposite end (i.e., “spraying tip”) of the capillary tube under high voltage through a Taylor cone electrospray, and guided towards collection.

In some embodiments, the regents to be reacted are loaded onto the apparatus neat, however, in many embodiments, the reagents are pre-dissolved in an appropriate solvent or solvents. In many embodiments, the solvent choices suited for the instant methods comprise (but are not limited to): water, methanol, ethanol, other polar solvents, other biocompatible solvents and solvents known to solubilize biomolecules, and any combination thereof. However, in some embodiments, the reaction is conducted anhydrously. In such embodiments, an anhydrous solvent may be used, such as, for example, acetonitrile, dimethylformamide, or another non-nucleophilic solvent.

In many embodiments, the Taylor cone spray is a fine aerosol/mist of highly charged microdroplets comprising much desolvated reagents, as most of the solvents evaporate upon electrospraying. In many embodiments, as the microdroplets containing the reagents desolvate upon electrospraying, the reagents' concentration greatly increases, thus accelerating the reaction progression. Accordingly, in many embodiments, solvents are chosen such that they have low enthalpies of vaporization in order to further augment the desolvation of the electrospray microdroplets. In many embodiments, the reaction conditions, including the extent of the microdroplet desolvation, are further optimized by determining and using the solubility parameters (i.e., the free-energy relationship of the solvents, gases, and reagents) to optimize the microdroplets' pathway to the collection vessel. In many embodiments, the voltage/ESI potential range applied to the microdroplets is optimized within 2 to 7 kV range for a given reaction to maximize the yield of the desired product. In some embodiments, the ESI ionization mode of the instant methods is positive, while in other embodiments, it is negative. In many embodiments, the temperature of the reaction media is adjusted along the microdroplets' pathway, as needed, to maximize the yield of the desired product.

In some embodiments, the collected reaction product or products are used as is, while in other embodiments, the products are subjected to one or more purification procedures, such as, for example, High-performance Liquid Chromatography (HPLC), prior to use. In some embodiments, purification and or the separation of the desirable biomolecules from unwanted solvents, intermediaries, or other byproducts is performed with tangential flow filtration (TFF). In some embodiments, the desired products are purified with the help of molecular sieves constructed from traditional materials, or new materials, such as graphene, or via any number of distillation techniques. However, in some embodiments, the collected reaction product or products are collected to be reacted as reagents in another reaction to obtain the final desired product. In yet other embodiments, the final desired product is only obtained upon acting in one of the following ways on the collected microdroplets: condensing, quenching, humidifying (if anhydrous), heating, cooling, and any combination thereof.

In some embodiments, techniques other than ESI are used to promote a reaction of choice, such as any other technique known to desolvate reaction reagents and or accelerate chemical reactions. For example, in some embodiments, sonochemistry is used to promote the synthesis of the desirable biomarkers, including fluoridation and radiolabeling of biomarkers. As another example, in some embodiments, tangential flow filtration (TFF) is used to desolvate reagents and accelerate the desired chemical reactions, including radiolabeling.

In many embodiments, the methods of the instant disclosure, especially when the methods are fluoridation methods, are further aided by utilizing a prosthetic moiety. In many such embodiments, the prosthetic moiety further directs and facilities the installation of a fluorine isotope, or another radionuclide, or another atom of functionality of choice onto a biomarker of choice. In many such embodiments, wherein the method is fluoridation, the prosthetic moiety is an FA. In many such embodiments the FA is a SiFA. In many other embodiments, the FA is HetSiFA. Accordingly, in many embodiments, the ultrafast radiolabeling kinetics afforded by FA moieties are utilized in addition to the already excellent reaction kinetics afforded by the implementation of ESI methods, thus, advantageously combining molecular design and reaction setup engineering to greatly accelerate and otherwise facilitate the manufacture of, for example, [¹⁸F]PET probes. In many embodiments, the improvements afforded by the instant methods are especially relevant to clinical uses, as they allow for much milder reaction conditions, better aligned with synthesis of viable complex biomarkers.

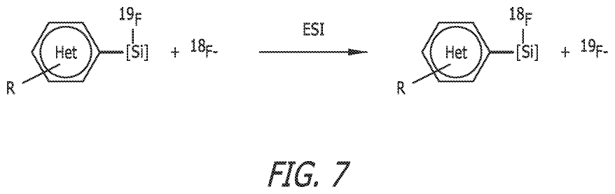

FIG. 7 illustrates the methods of the instant application, wherein ESI methods are combined with FA concepts according to many embodiments. In this example, a HetSiFA moiety is radiofluoridated with ESI help. In some embodiments, other additives, reagents, and catalysts are added to the reaction mixture as needed. For example, in the reaction illustrated in FIG. 7, in some embodiments, the ¹⁸F anion may be used as is, while in other embodiments a cryptand, such as 4,7,13,16,21,24-hexaoxa-1,10-diazabicyclo[8.8.8]hexacosane (K2.2.2), or another activating agent may be employed to further drive the reaction forward. In addition, in many embodiments, any or all reaction reagents are dried, purified, or otherwise manipulated in any way helpful in promoting their desired reactivity and overall reaction progress. Furthermore, in many embodiments, the source of a radionuclide used in the radiolabeling methods of the instant application, including ¹⁸F anion, may be a cyclotron/particle accelerator, or another reactor, or any other suitable source.

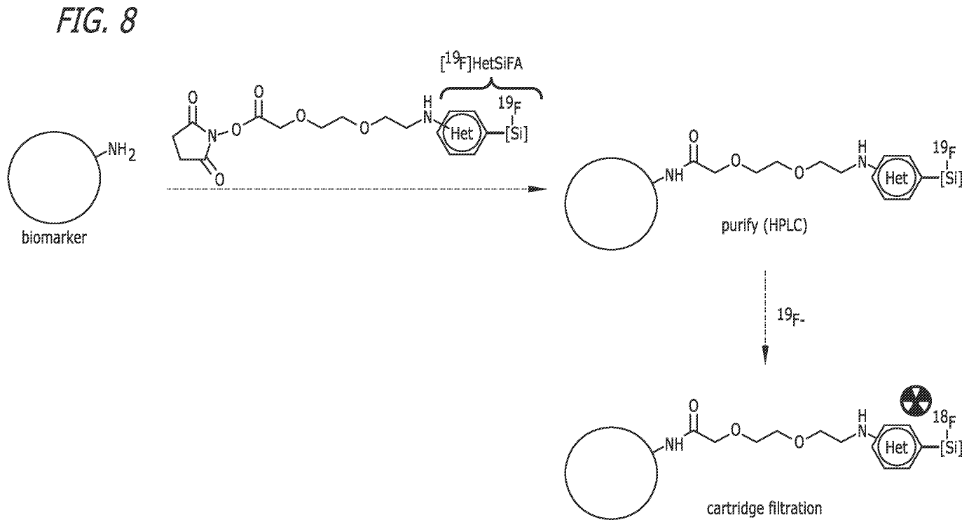

In some embodiments, wherein a prosthetic moiety is implemented in addition to ESI for facilitating fluoridation or radiolabeling, the prosthetic moiety is radiolabeled first and then attached to a desired biomarker via any suitable method, including via any pre-installed linker suitable for the chosen biomarker. However, in other embodiments, the prosthetic moiety is pre-installed into the biomarker prior to ESI treatment, and the fluoridation or radiolabeling according to the instant methods is the direct, final-stage fluoridation or radiolabeling of the biomarker, as, for example, illustrated in FIG. 8. In this example, a biomarker/biological targeting vector of choice, such as, for example, a protein, a peptide, an antibody, or another small biomolecule, or biopolymer, is first conjugated to a HetSiFA prosthetic carrying a labile “cold” ¹⁹F via a reaction between the biomarker's amine group and HetSiFA moiety's succinimide functionalized linker, followed by, for example, HPLC purification, and radiolabeling with ¹⁸F according to the microdroplet isotopic exchange methods of the instant disclosure. Finally, a cartridge filtration may be needed to maximize the yield and purity of the desired radiotracer for clinical applications. However, in many embodiments, performing the final-stage isotopic exchange reaction using ESI according to the instantly disclosed methods avoids the need for HPLC or other purifications, and, therefore, further enhances the efficiency and speed of the production of useful radiolabeled biomarkers. In many embodiments, combining the ESI and radiolabeling prosthetics approaches, according to the instant methods, allows for development of completely new classes of radiotracers, including of new targeted PET probes, for imaging of a variety of disease classes. In many embodiments, the combination approach is especially fit for clinical setting, and allows for rapid and efficient, on-demand production of radiopharmaceuticals, including established and new [¹⁸F]PET radiotracers.

In some embodiments, HetSiFA prosthetic moieties are prepared according to the synthetic methods described by Toutov et al., in Nature, 2015, 518:80-84, the disclosure of which is incorporated herein by reference, utilizing potassium tert-butoxide as the silylation catalyst.

In many embodiments, the ESI-assisted IEX methods of the instant disclosure, including, for example, ¹⁹F/¹⁸F exchange on a SiFA prosthetic, is performed on a platform such as a commercial radiosynthesizer, an in-house developed microfluidic TFPEcoated chip. However, in some embodiments, the instant methods are performed as a manual procedure in a sealed glass vial. In some embodiments, the instant disclosure is directed to a kit for ESI-assisted isotopic exchange radiolabeling, for example, for ¹⁸F/F IEX of SiFAs.

However, in many embodiments, especially in the instances when the addition of a prosthetic moiety, such as SiFA or HetSiFA, to a biomarker of choice unacceptably affects its pharmacokinetics, ESI, or another technique that allows to effectively concentrate aqueous solutions of clinically-relevant radioisotopes in situ, is directly applied to facilitate and or accelerate radiolabeling of the desired biomarkers and or minimize use of additional reagents or catalysts. For example, in many embodiments, ESI radiofluoridation is performed on substrates comprising a reactive site for fluoride. In such embodiments, application of ESI according to the instant methods greatly improves the reaction kinetics, as compared to conducting the same reaction in bulk solution, or even under microfluidic conditions. As another example, in many embodiments, ESI-assisted radiolabeling according to the instant methods activates the radionuclide without additional reagents, e.g., without addition of expensive crown ethers, such as K2.2.2 cryptand, which are typically required for efficient radiofluoridation.

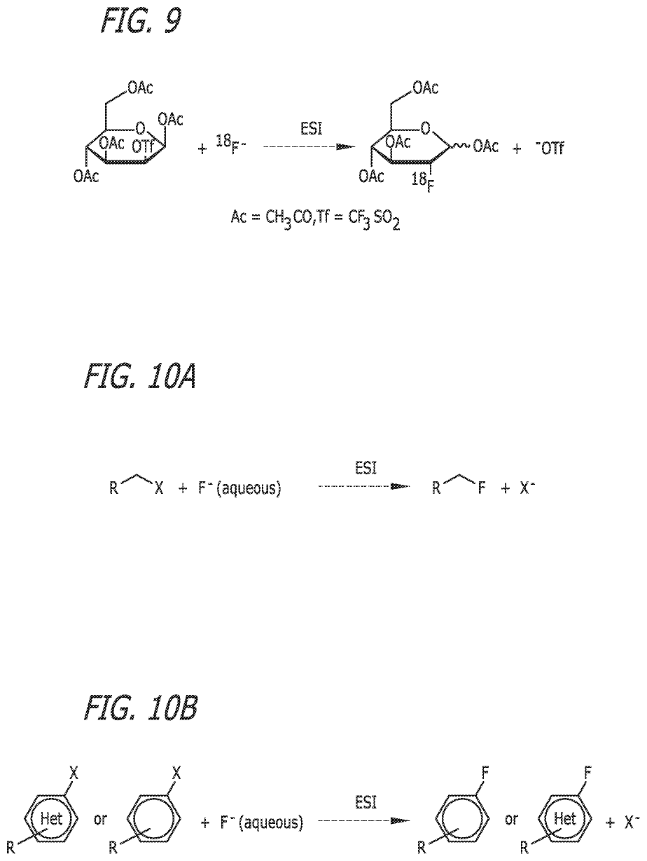

As a more specific example, FIG. 9 illustrates ESI-assisted radiofluoridation of protected fluorodeoxyglucose according to many embodiments of the instant application. Here, the source of ¹⁸F anion may be a cyclotron, or any other suitable source, and ¹⁸F anion may be pre-activated, for example, by addition of a crown ether, or manipulated in any other way prior to use to promote its installation onto the sugar, however, the instant methods are intended to eliminate or at least minimize such reagent activation requirements typically employed in bulk solution reactions.

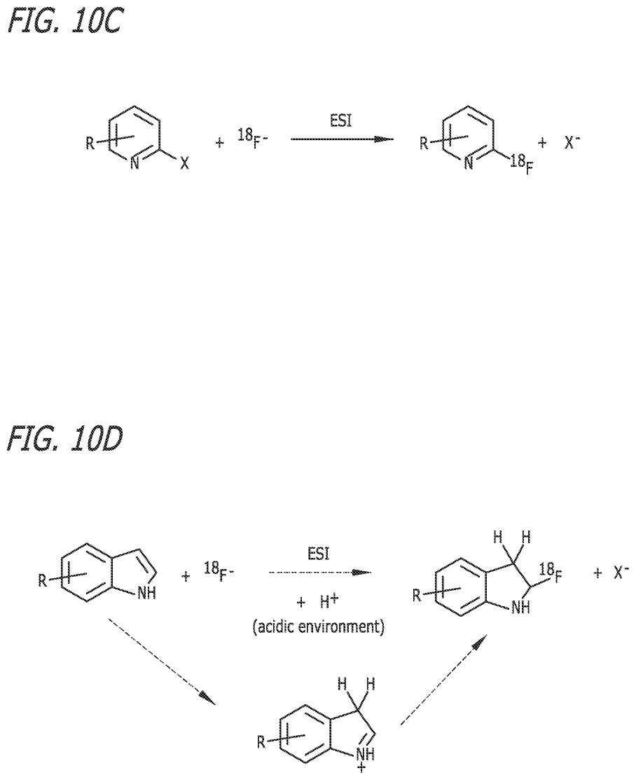

In many embodiments, the instant methods allow for direct fluoridation of C—X bonds, wherein X is a leaving group displaceable by a fluorine anion. More specifically, the electrospray microdroplets of the instant methods offer unique localized reaction environment, including acidic nature of the environment, and other advantageous features that may aid in promoting fluoridation of C—X bonds. For example, FIGS. 10A through 10D provide various illustrative examples of chemistries that might benefit from ESI-assisted methods of the instant disclosure. More specifically, FIG. 10A illustrates a direct fluoridation of an aliphatic molecule according to many embodiments of the instant methods, while FIG. 10B illustrates the same for aromatic and heteroaromatic molecules. Here, R in all FIGS. 10A through 10D may be any biomolecule, or any other molecule, or biomarker, or a part thereof; X may be, but is not limited to any of the following functionalities: OR′, NR′₂, N⁺R′₃, NC, NO₃, NO₂, OTs, SR′, C(O)R′, OC(═O)R′, OTf, OMs, OSO₃R′, a halogen, including “cold” ¹⁹F, or any other leaving group displaceable by an F anion, wherein each R′, in turn, is, independently, any one of the following functionalities: H, optionally substituted C₁₋₆ alkyl, C₁₋₆ haloalkyl, C₂₋₆ alkenyl, C₂₋₆ alkynyl, aryl, heteroaryl, and any combination thereof; and wherein F⁻ is any anionic isotope of fluorine. Furthermore, FIG. 10C more specifically shows radiofluoridation of a pyridine according to the instant methods, wherein, X is, for example, a halogen, or any other leaving group such as listed for X above. In addition, FIG. 10D shows radiofluoridation of an indole according to the instant methods. In many embodiments, placing molecules prone to protonation, such as indole, in the acidic environment afforded by ESI microdroplets produces predictable intermediates (FIG. 10D, bottom), and therefore, enhances yields of the desired products. As such, in many embodiments, various aspects of the microdroplet-afforded environment, such as, for example, high reagent concentration, acidity, and charge, are judiciously utilized to advantageously affect the progress of the desired chemical reaction, including the reaction's regio- and stereo-selectivity, to maximize the yield of the desired products, while minimizing the side reactions, and minimizing or eliminating the need for purification.

In many embodiments, the instant methods allow for direct fluoridation of Si—X and B—X bonds, wherein X is a leaving group displaceable by a fluorine anion, including: H, OR′, NR′₂, N⁺R′₃, NC, NO₃, NO₂, OTs, SR′, C(O)R′, OC(═O)R′, OTf, OMs, OSO₃R′, a halogen, including “cold” ¹⁹F, or any other leaving group displaceable by an F anion, wherein each R′, in turn, is, independently, any one of the following functionalities: H, optionally substituted C₁₋₆ alkyl, C₁₋₆ haloalkyl, C₂₋₆ alkenyl, C₂₋₆ alkynyl, aryl, heteroaryl, and any combination thereof.

### EXEMPLARY EMBODIMENTS

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric.

### Example 1: Radiofluoridation According to the Instant Methods

In many embodiments, a target molecule to be endowed with F atom is first identified and provided. The target molecule may be, for example, a biomarker to be radiolabeled for PET application, such as, for example, [¹⁹F]-FDG; or, as another example, a radiolabeling prosthetic moiety, such as a [¹⁹F]HetSiFA-carrying molecule to be radiolabeled with ¹⁸F for later conjugation to a PET biomarker, or, as yet another example, a biomarker pre-outfitted with a [¹⁹F]HetSiFA moiety for direct ¹⁸F-radiolabeling. In some embodiments, the target molecule is used neat, while in other embodiments, it is first dissolved in an appropriate solvent. Example of appropriate solvents compatible with the instant methods include: water, methanol, ethanol, other polar solvents, other biocompatible solvents and solvents known to solubilize biomolecules, anhydrous solvents, including acetonitrile and other non-nucleophilic solvent, and any combination thereof. In some embodiments, the target molecule is further combined with any number of stabilizing reagents. In some embodiments, the target molecule is treated to adjust its acidity or basicity.

Next, in many embodiments, a solution of ¹⁸F⁻ anion is provided and combined with the solution comprising the target molecule to form a reaction mixture. In some embodiments, ¹⁸F⁻ is provided as an aqueous solution. In some embodiments, ¹⁸F⁻ is dissolved is an anhydrous solvent, or some other non-nucleophilic solvent. In many embodiments, ¹⁸F⁻ is produced by a cyclotron, however, it may also be acquired from any other source. In many embodiments, the reaction mixture comprises reactants in stoichiometric proportion to minimize the need for post-production purification. For example, for the radiofluoridation of [¹⁹F]-FDG or [¹⁹F]HetSiFA moiety (by itself or attached to the intended biomarker), any of these target molecules is combined with ¹⁸F⁻ anion in 1:1 ratio. In some embodiments, wherein it is beneficial to have an above stoichiometric amount of one of the reactants, the appropriate adjustments are made to increase the yield of the desired product, or to provide other desirable outcomes.

In many embodiments, the reaction mixture is next injected into the capillary tube of an ESI apparatus. In many embodiments, a high voltage potential is applied to the reaction mixture, such that the reactants are charged. In many embodiments, the high voltage potential is in the +2 kV to +7 kV range, or in the −2 kV to −7 kV range, or even outside these ranges. In many embodiments, the voltage potential range is 3.5 kV to 5 kV. In many embodiments, the high voltage potential range is specifically optimized for the chosen reaction to maximize the yield of the desired product. In many embodiments, the high voltage potential is transferred to the reactants and stays with the reactants as the reaction mixture enters the ESI sprayer and is vaporized to form a mist of charged microdroplets containing the reaction mixture's reactants.

In many embodiments, as the reaction mixture is nebulized by the ESI sprayer and the solvents are evaporated from the microdroplets, the desired reaction is accelerated. Accordingly, in many embodiments, the parameters affecting the production of the microdroplets are adjusted as described herein to promote continuous desolvation process that further favors acceleration of the desired reaction. For example, in many embodiments, the solvent evaporation is promoted by the injection of an inert or otherwise non-reactive gas into the ESI sprayer. In many embodiments, the inert or otherwise non-reactive gas is used both to promote the desolvation of the reagents and, thus, accelerate the desired reaction, and, also, to guide the microdroplets to the collection vessel. In some embodiments, the inert or otherwise non-reactive gas is selected from (but not limited to) the group consisting of: any noble gas, nitrogen, carbon dioxide, and any combination thereof. In many embodiments, the inert or non-reactive gas is argon. In addition, in many embodiments, solvent evaporation is further promoted by drawing a partial vacuum within the ESI apparatus. Table 1 below provides examples of several reaction parameters that can be advantageously adjusted within the provided value ranges to drive the desired reaction forward.

TABLE 1

| Parameter | Range | |
|---|---|---|
| ESI ionization mode | positive and negative | |
| ESI potential | 3500-5000 | Volts |
| Nebulizing gas (NBG) temperature | 25-300° | C. |
| Spray flow rate | 10-1000 | min |

In many embodiments, next, the reactants-loaded microdroplets/effluent travel from the ESI sprayer to a collection vessel. In such embodiments, the length and trajectory of the microdroplets are adjusted to allow for the most efficient desolvation process to occur in order to maximally accelerate the desired reaction. However, in some embodiments, the desired reaction occurs (or is finalized) only in the collection vessel. In many such embodiments, the final desired product is only obtained upon acting in one of the following ways on the collected microdroplets: condensing, quenching, humidifying (if anhydrous), heating, cooling, and any combination thereof. In many embodiments, the radiolabeled product accumulates in the collection vessel and is ready for use, including as a reactant in another reaction, or a as a patient injectable radiotracer, when sufficient amount has, thus, accumulated.

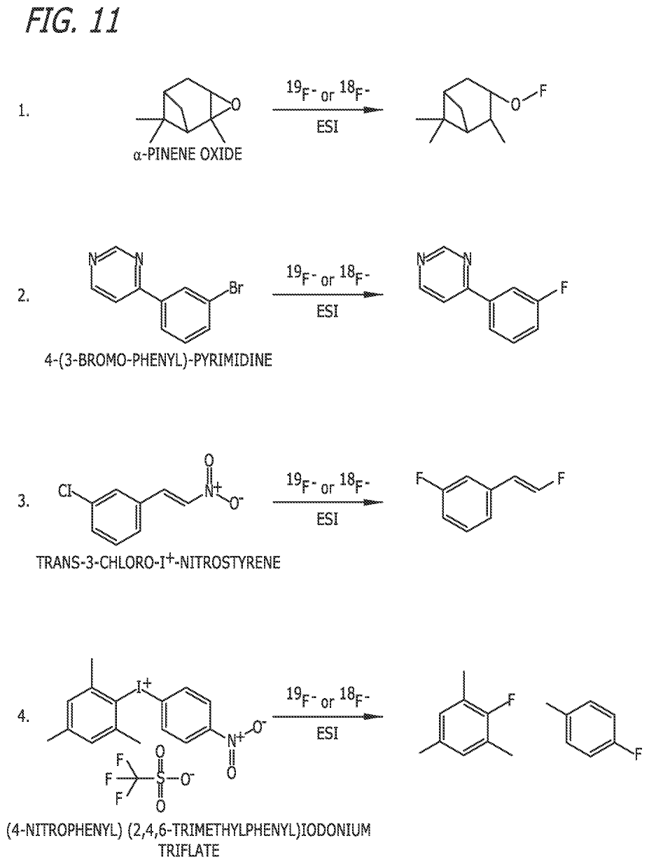

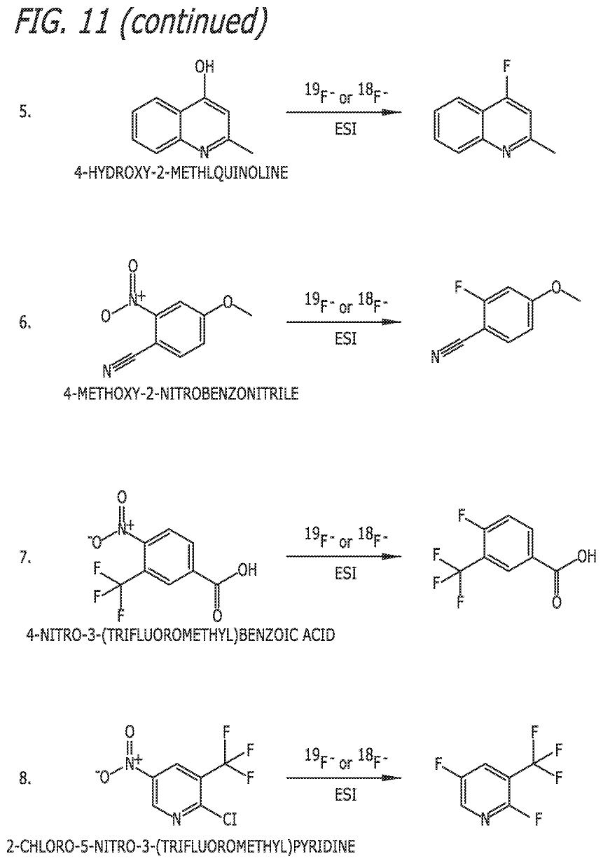

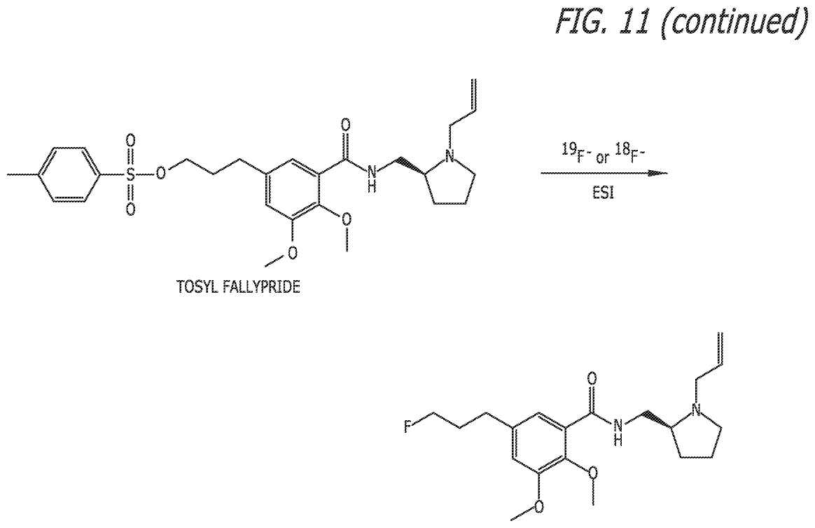

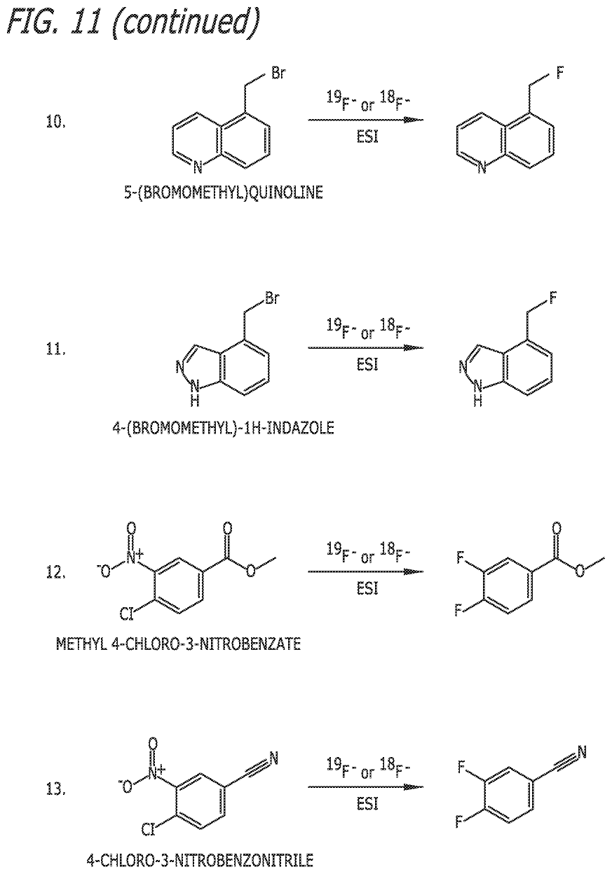

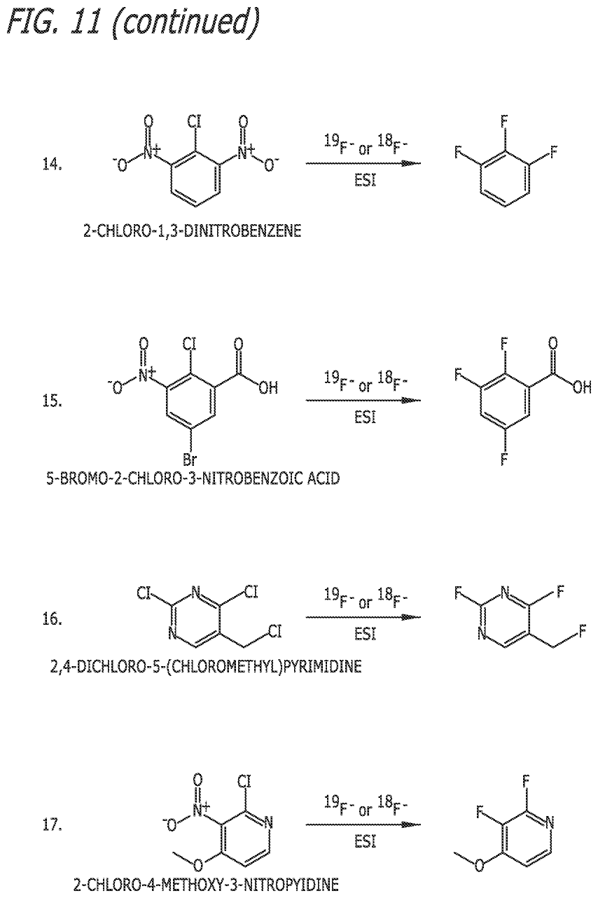

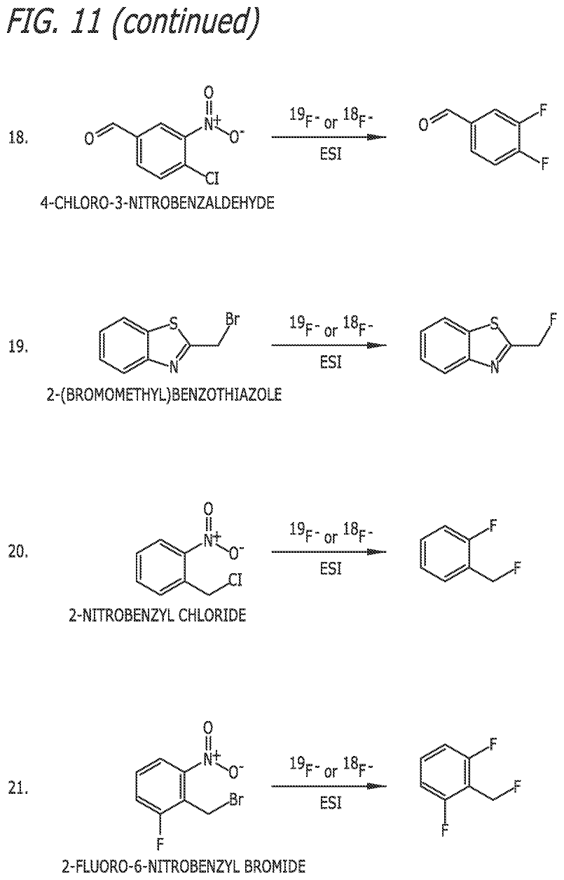

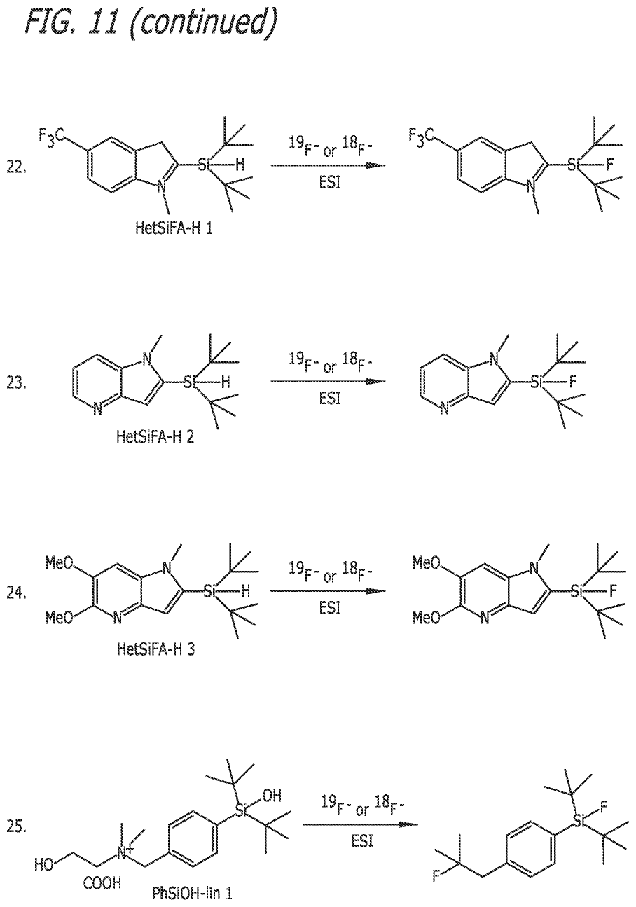

FIG. 11 provides a number of specific fluoridation examples illustrating many embodiments of the instant fluoridation methods, including ESI-assisted fluoridation of SiFA (reaction 25) and HetSiFA (reactions 22-24) moieties. In all these examples, ¹⁹F and ¹⁸F are used interchangeably.

### Example 2: Preparation of a Reaction Mixture for Radiofluoridation According to the Instant Methods

In some embodiments, a fluoridation reaction mixture is prepared according to the following steps:
1) a 20 mL saturated mixture of a fluoride salt (e.g., KF and or CsF) in a polar solvent (e.g., dimethylformamide and or acetonitrile) is prepared (the dissolved concentration is 32 μg/mL) and after vortex mixing is set aside to let the undissolved salt precipitate;
2) a 2 mg/mL solution of a target molecule in a chosen solvent (e.g., in the same solvent as used to dissolve the fluoride salt in step (1), or whichever solvent best dissolves the target molecule) is prepared (2 mL total volume);
3) 6 mL of the supernatant solution of F⁻ from step (1) is combined with 200 μL of the target molecule solution from step (2) solution and vortex-mixed for 30 seconds.

### Example 3: Examples of Substrate-Specific Reaction Conditions According to the Instant Fluoridation Methods

Table 2 below provides examples of reaction conditions optimized for the ESI-assisted radiofluoridation of some of the target molecules (substrates) shown in FIG. 11. The conversion values for this table were calculated based on the mass spectrometry analysis of the reaction mixture products using the equation provided below, wherein RPA represents relative peak area:

$$conversion ( \% ) = \frac{R P {A}_{product}}{( R P {A}_{product} + R P {A}_{Substrate} )} \times 100$$

TABLE 2

| | | | | | ESI | NBG | Spray |
|---|---|---|---|---|---|---|---|
| | Conversion | Fluoride | | ESI | potential | Temp. | flow rate |
| Substrate | (%) | salt | Solvent | mode | (V) | (° C.) | (μL/min) |
| 8 | 47.5 | KF | DMF | Negative | 3500 | 25 | 50 |
| 9 | 45.00 | CsF | CH3CN | Positive | 3500 | 200 | 4 |
| | | | (+10 vol/vol % DI- | | | | |
| | | | water | | | | |
| 16³ | 94.64 | CsF | DMF | Negative | 3500 | 25 | 40 |
| 23⁴ | 44.22 | CsF | DMF | Positive | 3500 | 25 | 50 |
| 24⁵ | 0.93 | CsF | CH3CN | Positive | 3500 | 25 | 250 |
| 25⁶ | 59.50 | CsF | CH3CN | Positive | 5000 | 300 | 450 |

The order of steps of the methods described herein may occur in a variety of sequences, and the various steps of the methods described herein can be combined with other steps, interlineated with the stated steps, and/or split into multiple steps. Similarly, the apparatus elements have been described functionally, and can be embodied as separate components or can be combined into components having multiple functions. Furthermore, obvious modifications and alterations to the described embodiments are available to those of ordinary skill in the art.

### DOCTRINE OF EQUIVALENTS

This description of the invention has been presented for the purposes of illustration and description. It is not intended to be exhaustive or to limit the invention to the precise form described, and many modifications and variations are possible in light of the teaching above. The embodiments were chosen and described in order to best explain the principles of the invention and its practical applications. This description will enable others skilled in the art to best utilize and practice the invention in various embodiments and with various modifications as are suited to a particular use. The scope of the invention is defined by the following claims.

1. A method for electrospray ionization-assisted fluoridation, comprising
providing a target molecule for a fluorine isotope to be installed on and optionally dissolving the target molecule in a first solvent to obtain a target molecule solution;
providing a fluorine anion and dissolving the fluorine anion in a second solvent to obtain a fluoride solution;
mixing the target molecule solution with the fluoride solution to obtain a reaction mixture;
optionally adding one or more reagents for activation of either the fluorine anion, or the target molecule, or both to the reaction mixture;
applying an ionization potential and mode to the reaction mixture, and nebulizing the reaction mixture under the ionization potential and mode to create a plurality of microdroplets comprising the reaction mixture;
allowing the target molecule and the fluorine anion to chemically interact within the plurality of microdroplets to produce a reaction product, wherein the plurality of microdroplets continuously desolvates until collection;
collecting the plurality of microdroplets comprising the reaction product in a collection vessel;
to obtain a fluoridation product of the chemical reaction between the target molecule and the fluorine anion in clinical-quality yield and purity.

2. The method of claim 1, wherein the fluorine anion is selected from the group consisting of ¹⁹F⁻, ¹⁸F⁻.

3. The method of claim 1, wherein the first and second solvent are, independently selected from the solvent list consisting of: water, methanol, ethanol, other polar solvent, other biocompatible solvent known to solubilize biomolecules, dimethylformamide, acetonitrile, another anhydrous solvent, another non-nucleophilic solvent, and any combination thereof.

4. The method of claim 1, wherein the first solvent and the second solvent are the same solvent.

5. The method of claim 1, wherein the ionization potential is in the range from 2,000 Volts to 7,000 Volts.

6. The method of claim 1, wherein the ionization potential is in the range from 3,500 Volts to 7,000 Volts.

7. The method of claim 1, wherein the ionization mode is selected from the group consisting of: positive, negative.

8. The method of claim 1, wherein additional means are provided to assist with desolvation of the plurality of microdroplets, and or to guide a flow of the plurality of microdroplets towards the collection vessel, and or to otherwise accelerate the chemical reaction within the plurality of microdroplets.

9. The method of claim 8, wherein the additional means comprise one or more means selected from the list consisting of: utilizing a nebulizing gas heated to 25-300° C.; heating or cooling the reaction mixture at any point of the reaction; condensing, quenching, and or humidifying the plurality of microdroplets at collection; and any combination thereof.

10. The method of claim 1, wherein the collected plurality of microdroplets are purified to isolate the reaction product prior to use.

11. The method of claim 1, wherein the target molecule is a Fluoride Acceptor comprising a linker moiety for conjugation with a final target.

12. The method of claim 11, wherein the Fluoride Acceptor is a prosthetic molecule selected from the group consisting of: a SiFA, a HetSiFA.

13. The method of claim 1, wherein the target molecule comprises a Fluoride Acceptor functionality.

14. The method of claim 13, wherein the Fluoride Acceptor functionality is a prosthetic functionality selected from the group consisting of: a SiFA functionality, a HetSiFA functionality.
